(19) United States
(12) Patent Application Publication
(10) Pub. No.: US 2025/0381066 A1
(43) Pub. Date: Dec. 18, 2025

(54) APPLICATOR FOR HANDLING A DRY OCULAR HEALTH PRODUCT AND PRIMARY PACKAGE CONTAINING SUCH PRODUCT

(71) Applicant: Alcon Inc., Fribourg (CH)

(72) Inventors: Felix Brinckmann, Roßdorf (DE); Justin Robert Barla, Westworth Village, TX (US); Christoph Ludwig, Brensbach (DE); Sushant Muchhala, Grand Prairie, TX (US); Ulrike Rostek, Offenbach (DE); Nils Schweizer, Bad König (DE)

(21) Appl. No.: 19/234,507

(22) Filed: Jun. 11, 2025

Related U.S. Application Data
(60) Provisional application No. 63/659,595, filed on Jun. 13, 2024.

(51) Int. Cl. A61F 9/00; B65B 5/04; B65B 7/28; B65D 75/32

(52) U.S. Cl. CPC A61F 9/0017; A61F 9/0026; B65B 5/04; B65B 7/2842; B65D 75/326

(57) ABSTRACT

An applicator for gripping and removing a dry ocular health product from a storage container and for placing the removed dry ocular health product on the eye of a patient, the applicator comprising:

a handling portion for handling the applicator; and

a tip portion connected to the handling portion, wherein at least a tip portion of the applicator is made from an electrostatically chargeable material to make the dry ocular health product adhere to the tip portion due to electrostatic forces exerted by the tip portion of the applicator on the dry ocular health product, and for subsequently releasing the dry ocular health product from the tip portion on the eye of the patient by means of attraction forces caused by a tear film in the eye of the patient.

### FIELD

The present invention generally relates to the field of providing, handling and applying a dry ocular health product. More particularly, the invention relates to an applicator for handling a dry ocular health product, a primary package containing a dry ocular health product and a said applicator, and to a method of manufacturing a said primary package.

### BACKGROUND

Dry eye disease is a condition that occurs when tears are not able to provide adequate lubrication for the eyes. For example, dry eyes may occur if the eyes do not produce enough tears or if they produce poor-quality tears. This may lead to inflammation and damage of the eye's surface. Treatments for dry eyes may help the patient feel more comfortable.

Treatments for dry eyes typically include the use of eye drops, also called artificial tears. Alternative treatments include the placement of ocular health products in the eye which may provide relief to the patient. A known ocular health product to be placed in the eye is sold under the trademark LACRISERT®. This ocular health product is a hydroxypropyl cellulose ocular insert that looks like a clear grain of rice. To apply the insert, the patient is required to pull a corner of the lower eyelid down to create a small pocket in the corner of the lower eyelid. The insert is then placed in the so formed pocket with the aid of a rigid applicator comprising two rigid tips and a recess formed between these tips. The insert is adhered to the applicator in the recess. Care must be taken during insertion that the rigid tips do not contact the eye itself to avoid discomfort or harm to the patient's eye. Once being placed in the pocket, the insert dissolves slowly and releases a substance that is used in eye drops to lubricate the eye.

Due to the rigidity of the applicator, in particular the two tips defining the recess in which the insert is adhered to the applicator for insertion into the pocket created in the corner of the lower eyelid, patients may feel uncomfortable, especially when the applicator approaches the lower eyelid, as they may be afraid of inadvertently touching the eyeball with the two rigid tips of the applicator.

It is therefore an object to overcome the afore-mentioned disadvantages and to provide an alternative applicator for applying an ocular health product that avoids contacting the eye upon applying the ocular health product, and thus to increase patient comfort.

### SUMMARY OF THE INVENTION

In accordance with a first aspect of the present invention, an applicator for gripping and removing a dry ocular health product from a storage container and for placing the removed dry ocular health product on the eye of a patient is suggested. The applicator comprises:
a handling portion for handling the applicator; and
a tip portion connected to the handling portion, for gripping the ocular health product from the storage container and for subsequently releasing the gripped ocular health product from the tip portion on the eye of the patient, wherein at least the tip portion of the applicator is made from an electrostatically chargeable material to make the dry ocular health product adhere to the tip portion due to electrostatic forces exerted by the tip portion of the applicator on the dry ocular health product, and for subsequently releasing the dry ocular health product from the tip portion on the eye of the patient by means of attraction forces caused by a tear film in the eye of the patient.

In accordance with one aspect of the applicator according to the invention, the material the applicator is made of is a soft material that is capable of deforming upon contacting the eye.

In accordance with another aspect of the applicator according to the invention, the electrostatically chargeable material the tip is made of is silicone.

In accordance with one example, the whole applicator is made of silicone.

In accordance with still another aspect of the applicator according to the invention, the dry ocular health product has a flat surface having an ocular health product surface area, and wherein the tip portion of the applicator has a flat surface having tip portion surface area that is substantially congruent with the ocular health product surface area.

In accordance with yet another aspect of the applicator according to the invention, the handling portion is elastically deformable in a direction transverse to a direction of the connection of the tip portion and the handling portion.

In accordance with a second aspect of the invention, a primary package containing a dry ocular health product and an applicator as described above is suggested. The primary package comprises a fluid-tight storage container having a cavity accommodating the dry ocular health product and the applicator. The primary package further comprises a fluid-tight cover foil sealed along a sealing portion of the storage container, with the sealing portion completely extending around the cavity accommodating the dry ocular health product and the applicator. The dry ocular health product is arranged in the cavity at the location where the tip portion of the applicator is arranged.

In accordance with one aspect of the primary package according to the invention, the dry ocular health product is a dried polymer gel film to be dissolved upon being placed on the eye.

In accordance with another aspect of the primary package according to the invention, the fluid-tight storage container is made of a fluid-tight foil, for example an aluminum foil, and the cavity is formed in the fluid-tight foil through deep-drawing or thermoforming, or through combinations thereof.

In accordance with a further aspect of the primary package according to the invention, the dry ocular health product is transparent or translucent, and the fluid-tight cover foil is opaque and colored.

In accordance with a third aspect of the invention, a method of manufacturing a primary package as discussed above is suggested. The method comprises:
providing an applicator for gripping and removing a dry ocular health product from a fluid-tight storage container, and for placing the removed dry ocular health product on the eye of a patient, the applicator comprising a handling portion for handling the applicator and a tip portion connected to the handling portion, the tip portion for gripping the ocular health product;
providing a fluid-tight storage container comprising a cavity for accommodating the applicator and the dry ocular health product, and having a sealing portion completely extending around the cavity;
providing a fluid-tight cover foil to be sealed to the storage container along the sealing portion, with a surface of the cover foil to be sealed to the storage container along the sealing portion facing upwards;
dispensing a predetermined amount of a liquid ocular health product on a predetermined area of the surface of the cover foil that is to be sealed to the storage container along the sealing portion;
drying the predetermined amount of the liquid ocular health product to obtain a dry ocular health product that is arranged on the predetermined area of the surface of the cover foil that is to be sealed to the storage container along the sealing portion;
placing the applicator in the cavity of the storage container in a manner such that the applicator is contained therein frictionally engaged by inner walls of the cavity;
placing the storage container with the frictionally engaged applicator contained therein on the cover foil, with the sealing portion of the storage container facing towards that surface of the cover foil on which the dry ocular health product is arranged, and with the cavity arranged to accommodate the dry ocular health product at the location where the tip portion of the applicator is arranged; and
sealing the cover foil to the storage container along the sealing portion to form the primary package.

In accordance with one aspect of the method according to the invention, the applicator is an applicator having a handling portion that is elastically deformable in a direction transverse to a direction of the connection of the tip portion and the handling portion, and the method further comprises the step of folding or compressing the handling portion of the applicator during placing the storage container with the frictionally engaged applicator contained therein on the cover foil.

A dry ocular health product referred to herein is an ocular health product that may or may not contain a liquid, but does not have any wet surfaces. It is particularly sensitive to liquids, moisture or vapor, and must therefore be stored in the storage container in a manner such that the dry ocular health product does not come into contact with liquids, moisture or vapor during storage. One example for a dry ocular health product is a polymer gel film. In one embodiment (without being limited thereto), the dry ocular health product is a product that provides relief to patients suffering from dry eye disease.

The present invention offers a number of advantages. First of all, due to the tip portion of the applicator being made from an electrostatically chargeable material (one example for such material being silicone which is a material that is widely accepted by authorities in charge of approval of materials in the healthcare sector) the dry ocular health product can be easily gripped from a storage container by electrostatic forces acting on the ocular health product and making the dry ocular health product adhere to the tip of the applicator. That is, for picking the dry ocular health product up from the storage container the tip must be moved towards the dry ocular health product until the distance of the tip is sufficiently small to allow the electrostatic forces to attract the dry ocular health product and make it adhere to the tip. No mechanical gripping action is required. Alternatively, the dry ocular health product may already adhere to the gripper at the time of removing the gripper (together with the ocular health product) from the storage container, so that no (electrostatic) gripping action is necessary.

The dry ocular health product may then be transported (adhered to the tip) to the eye of the patient, and may be placed directly on the eye of the patient (i.e. on the surface of the eyeball of the patient). It is not necessary to form a pocket in the corner of the lower eyelid of the patient and to insert the dry ocular health product into such pocket, and the dry ocular health product is no longer adhered in a recess between two rigid tips of the applicator so that particular care must be taken not to contact the eyeball with such rigid tips. Rather, at the time the tip of the applicator approaches the eyeball of the patient the attraction forces caused by the tear film in the eye of the user make the dry ocular health product getting released from the tip of the applicator on the eye of the user (i.e. to the surface of the eyeball). This release of the dry ocular health product from the tip of the applicator occurs through flipping of the dry ocular health product away from the tip of the applicator on the eye of the patient (due to the attraction forces caused by the tear film in the eye of the patient) prior to the tip of the applicator contacting the surface of the eyeball. This is because the dry ocular health product adhering to the tip of the application faces the surface of the eyeball of the patient. For example, the dry ocular health product may be a dried polymer gel film that is dissolved once it is placed on the eye. Even though being a simplification, the dry ocular health product may be viewed as a dried lacrimal fluid in form of a dried polymer gel film that is placed on the eye and that dissolves once coming into contact with the tear film of the eye. Due to its sensitivity to moisture, the dry ocular health product must be stored in a manner so as to be protected from contact with moisture prior to its use.

It is favorable when the electrostatically chargeable material of the applicator is a soft material that is capable of deforming in case the tip of the applicator may inadvertently contact the eye of the patient nevertheless. In case of such inadvertent contact with the eye the patient does not experience a huge discomfort, as opposed to a rigid applicator where the discomfort experienced by the patient may easily reach an unacceptable level. One practical example for such electrostatically chargeable soft material is silicone. Silicone is a material that is widely accepted by authorities in charge of approval of materials in the healthcare sector. Either the tip or, even better, the whole applicator may be made of silicone. Silicone is also favorable with respect to reliable manufacturing of the applicator, as silicone allows to use well-established molding techniques.

The dry ocular health product (e.g. the dried polymer gel film) may, for example, be a disc having two flat ocular health product surfaces. One of these ocular health product surfaces faces the tip of the applicator, and it is then favorable if the tip surface is a flat surface that is substantially congruent with one of the ocular health product surfaces. The term ‘substantially congruent’ in this regard is intended to mean that the flat tip surface covers at least 60% of the ocular health product surface (in order to achieve a good adherence of the dry ocular health product to the tip of the applicator. Also, it may be favorable if the handling portion of the applicator is elastically deformable in a direction transverse to a direction of the connection of the tip portion and the handling portion. More frankly speaking, this means that the handling portion may be foldable or compressible in a vertical or horizontal direction (which is at the same time transvers to the direction of the connection of the tip portion and the handling portion). This allows the handling portion to be folded or compressed at the time of packaging, and holding it folded or compressed in the package as long as the package is sealed. Once the package is opened, the handling portion may unfold or expand so that it can be easily gripped and taken out of the package.

The primary package according to the invention comprises a fluid-tight storage container that has a cavity accommodating both the applicator as well as the dry ocular health product (e.g. a dried polymer gel film). The dry ocular health product is arranged in the cavity at the location where the tip portion of the applicator is arranged to make sure the dry ocular health product adheres to the tip of the applicator by the afore-described electrostatic forces. A fluid-tight cover foil is sealed along a sealing portion of the storage container, and this sealing portion completely extends around the cavity so that once the fluid-tight cover foil is sealed to the storage container the content of the cavity is hermetically sealed and protected from moisture or vapor entering the cavity. As mentioned, this is necessary due to the sensitivity of the dry ocular health product to moisture. To access the product, the patient may peel the foil off to gain access to the cavity and then grasp the handling portion of the applicator in order to remove the applicator and the dry ocular health product adhering to the tip portion from the cavity. Thereafter, the patient may place the dry ocular health product on the eye by approaching the eye with the dry ocular health product adhered to the tip portion facing towards the surface of the eyeball. As the dry ocular health product is arranged at a small distance from the surface of the eyeball, the dry ocular health product is getting released from the tip of the applicator due to the attraction forces caused by the tear film, and flips to the surface of the eyeball without the tip portion contacting the eye. Accordingly, the dry ocular health product is stored in a fluid-tight manner in the primary package and may be easily taken out from the package and placed on the eye without the applicator contacting the eye. And even if any portion of the applicator may inadvertently contact the eye, in case the applicator is made of a soft material the patient does not experience a huge discomfort.

The fluid-tight storage container may be made from a fluid-tight foil (e.g. aluminum) that allows deep-drawing or thermoforming of the foil, or combinations thereof, to form the cavity. Thus, the storage containers may be reliably manufactured by well-established techniques (here: deep-drawing and thermoforming), and the applicator as well as the dry ocular health product is well-protected from liquid, moisture and vapor. The fluid-tight foil the storage container is made of may be transparent/translucent or opaque, and may be colored or uncolored.

The fluid-tight cover foil may be transparent/translucent, too, or may be opaque, and may be colored or uncolored. It is advantageous, however, when the dry ocular health product is transparent or translucent, while the fluid-tight cover foil is opaque and colored. This is advantageous with respect to manufacturing of the primary package containing the dry ocular health product, as the colored an opaque cover foil allows for good visibility of the dry ocular health product arranged thereon during manufacture, as will be explained in more detail in the following.

In such method of manufacturing the afore-mentioned primary package, a predetermined amount of the liquid ocular health product is dispensed on a surface of the cover foil at a predetermined area thereof. This surface of the cover foil on which the liquid ocular health product is dispensed faces upwards and is that surface of the cover foil which is subsequently sealed to the storage container along the sealing portion of the storage container. The liquid ocular health product is then dried to obtain the dry ocular health product (e.g. the disk of polymer gel film) which is arranged on the predetermined area of the surface of the cover film. In case the dry ocular health product is transparent or translucent and the cover film is colored and opaque, this is advantageous inasmuch as the dry ocular health product is clearly visible due to the enhanced contrast between the dry ocular health product and the cover foil.

The applicator is placed into the cavity of the storage container such that it is frictionally engaged by the inner walls of the cavity. Thereafter, the storage container with the applicator contained therein is placed on the cover foil with the sealing portion of the storage container facing that surface of the cover foil on which the dry ocular health product is arranged. The cavity is arranged such that the ocular health product is accommodated in the cavity at the location where the tip portion of the applicator is arranged. Thereafter, the cover foil is sealed to the storage container along the sealing portion, thus forming the hermetically sealed primary package containing the dry ocular health product and the applicator in a fluid-tight manner protected against liquid, moisture and vapor.

In case the applicator comprises a handling portion that is foldable or compressible in a direction transverse to the direction of the connection of the tip portion and the handling portion, and in case during placing the storage container (e.g. the deep-drawn transparent or translucent plastic foil) on the cover foil the foldable or compressible handling portion is then folded or compressed, the sealed primary package contains the folded or compressed applicator. Upon opening the primary package, i.e. peeling the cover foil off, the handling portion (but not the tip portion to which the dry ocular health product adheres) may then unfold or expand so that it can be easily grasped by the patient.

### BRIEF DESCRIPTION OF THE DRAWINGS

Further advantageous aspects of the invention become apparent from the following description of embodiments of the invention with the aid of the drawings, in which:

FIG. 1 shows a perspective view of a first embodiment of an applicator according to the invention;

FIG. 2 shows a perspective view of the applicator of FIG. 1, with a disk-shaped dry ocular health product adhering to the tip portion of the applicator;

FIG. 3 shows a perspective view of a second embodiment of an applicator according to the invention;

FIG. 4 shows a perspective view of an embodiment of the fluid-tight cover foil of the primary package according to the invention;

FIG. 5 shows a perspective view of the cover foil of FIG. 4, with an amount of liquid ocular health product dispensed on a predetermined area of a surface of the cover foil;

FIG. 6 shows a perspective view of the cover foil of FIG. 4, with the dry ocular health product arranged on the predetermined area of the surface of the cover foil, obtained through drying of the predetermined amount of liquid ocular health product shown in FIG. 5;

FIG. 7 shows a perspective view of an embodiment of the storage container comprising a cavity containing the applicator shown in FIG. 1;

FIG. 8 shows a perspective view of the storage container shown in FIG. 7 turned upside-down and arranged above a cover foil having the dry ocular health product arranged on the predetermined area as shown in FIG. 6;

FIG. 9 shows a perspective view of an embodiment of an opened primary package, including the storage container in the cavity of which the applicator and the dry ocular health product are arranged, with the cover foil being peeled off to an extent such that the applicator is accessible for a patient;

FIG. 10 shows a perspective view of another embodiment of an opened primary package (without cover foil) including the storage container in the cavity of which a third embodiment of the applicator according to the invention is shown in a relaxed state, with the dry ocular health product adhered thereto;

FIG. 11 shows a perspective view of the cover foil suitable of the primary package the storage container of which shown in FIG. 10; and

FIG. 12 shows a perspective view of the storage shown in FIG. 10, with the third embodiment of the applicator arranged therein in an elastically deformed state.

### DESCRIPTION OF EMBODIMENTS

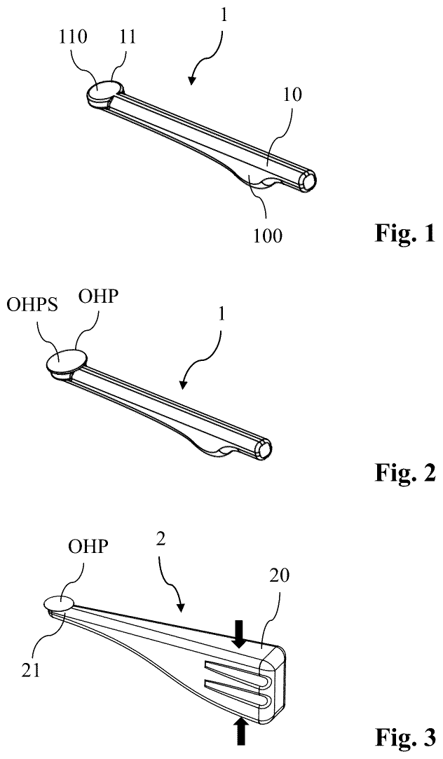

FIG. 1 shows a first embodiment of an applicator 1 according to the instant invention. The applicator comprises a handling portion 10 (proximal end) for handling the applicator 1. Applicator 1 further comprises a tip portion 11 (distal end) connected to the handling portion 10, for gripping a dry ocular health product and removing it from a storage container. Handling portion 10 comprises a tab 100 allowing the patient to reliably grasp the applicator 1 at the handling portion 10. Tip portion 11 comprises a flat surface 110 for gripping a dry ocular health product and making the dry ocular health product adhere to flat surface 110. At least the tip portion 11, but preferably the whole applicator 1, may be made of silicone. Silicone is a soft material that is elastically deformable and is easily electrostatically chargeable to make a dry ocular health product adhere to tip portion 11, in particular to flat surface 110 of tip portion 11. In addition, silicone is a material that is widely accepted by various authorities in charge of approval of materials in the healthcare sector.

FIG. 2 shows the applicator 1 of FIG. 1 with a dry ocular health product OHP having a shape of a disc having two flat surfaces OHPS (only that one facing upwards being visible), each having an ocular health product surface area. Dry ocular health product OHP may, for example, be a (transparent or translucent) polymer gel film. Flat surface 110 of tip portion 11 of applicator 1 (see FIG. 1) has a tip portion surface area that is substantially congruent with ocular health product surface area (i.e. covers at least 60% thereof). Dry ocular health product OHP adheres to tip portion 11 due to electrostatic forces between flat surface 110 of tip portion 11 and the corresponding flat surface OHPS (not visible) of ocular health product OHP.

Once dry ocular health product OHP, e.g. the polymer gel film, adheres to the tip portion of applicator 1 as shown in FIG. 2 i.e. to the flat surface 110 of tip portion 11 shown in FIG. 1), the applicator 1 may be moved by the patient towards the eye, with the free surface OHPS of dry ocular health product OHP (i.e. that surface of dry ocular health product OHP facing away from the flat surface 110 of tip portion 11 of applicator 1) facing towards the surface of the eyeball of the patient. At the time dry ocular health product OHP is arranged a small distance from the eyeball (still without touching the eye), due to attraction forces caused by the tear film in the eye dry ocular health product OHP releases from tip portion 11 of applicator 1 in a flipping movement towards the eye, and subsequently adheres to the eye of the patient where dry ocular health product OHP (here: the polymer gel film) dissolves.

FIG. 3 shows a second embodiment of an applicator 2 according to the invention. Like the first embodiment, this second embodiment of the applicator 2 comprises a handling portion 20 (proximal end) for handling the applicator 2. Applicator 2 further comprises a tip portion 21 (distal end) connected to the handling portion 20, for gripping the dry ocular health product. Unlike the first embodiment, the second embodiment does not comprise a flat surface at the tip portion 21. The handling portion 20 is elastically deformable, in this case foldable/compressible, in a direction transverse to the direction of the connection of the tip portion 21 and the handling portion 20. This direction of folding/compression is indicated by the unlabeled arrows in FIG. 3. Upon releasing compression, handling portion 20 may unfold/expand again to the state shown in FIG. 3.

Like the first embodiment, the applicator 2 may be made of silicone which is a soft material that is elastically deformable and is easily electrostatically chargeable to make the dry ocular health product adhere to the tip portion 21 (in this case the surfaces of the disc-shaped dry ocular health product OHP would be arranged perpendicular to the direction of the connection of the handling portion 20 and the tip portion 21 of applicator 2 (i.e. the longitudinal direction of applicator 2).

Once dry ocular health product OHP, e.g. the polymer gel film, adheres to the tip portion 21 of the applicator 2, the applicator 2 may be moved by the patient towards the eye, with the free surface OHPS of dry ocular health product OHP facing the surface of the eyeball of the patient. At the time dry ocular health product OHP is arranged a small distance from the eyeball (still without touching the eye), due to attraction forces caused by the tear film in the eye dry ocular health product OHP releases from the tip portion 21 in a flipping movement towards the eye, and subsequently adheres to the eye of the patient where dry ocular health product OHP (e.g. the polymer gel film) dissolves.

Combinations of features of the two embodiments of the applicator are conceivable as well. For example, an applicator may comprise a foldable/compressible handling portion (proximal end) as well as a flat surface at the tip portion (distal end).

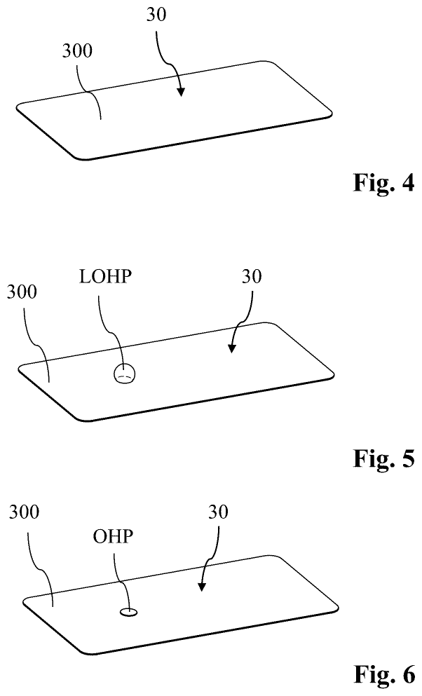

In the following, with the aid of FIG. 4 to FIG. 8 the method of manufacturing a primary package containing an applicator and an ocular health product is described. First of all, and as shown in FIG. 4, a fluid-tight cover foil 30 (which may not be penetrated by liquid, moisture and vapor) is provided. Fluid-tight cover foil 30 has a surface 300 that is to be sealed to a storage container along a sealing portion (this being described further below). Cover foil 30 may be a multi-layer construction that has a low-density polyethylene layer on the (inside) surface 300 (to be melted during sealing), and may further comprise an aluminum layer forming a barrier to liquid, moisture and vapor. Yet further, cover foil 3 may comprise additional plastic layers and print layers at the outside (facing downwards, not visible in FIG. 3). Cover foil 30 may be colored and opaque so as to make sure transparent or translucent dry ocular health product is well visible on cover foil 30, as becomes more evident from the following description.

Next, as shown in FIG. 5 a predetermined amount of a liquid ocular health product LOHP is dispensed on a predetermined area of surface 300 of cover foil 30. The predetermined amount of liquid ocular health product LOHP is then dried to form dry ocular health product OHP which is then arranged on the predetermined area on surface 300 of cover foil 30. This is shown in FIG. 6.

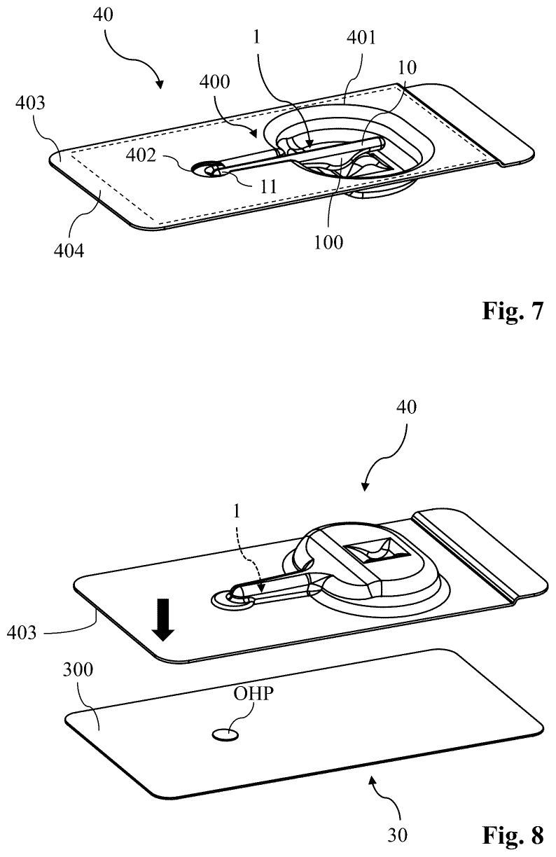

FIG. 7 shows a storage container 40 (shell) comprising a cavity 400 for accommodating both the applicator 1 as well as the dry ocular health product OHP. Cavity 400 comprises a first cavity portion 401 (proximal end) for accommodating the handling portion 10 of applicator 1. Applicator 1 is frictionally engaged in the cavity 400 by inner walls of the cavity 400, so that upon turning storage container 40 with applicator 1 contained therein upside-down applicator 1 does not fall down but remains in the cavity 400.

First cavity portion 401 is large enough to allow the patient to conveniently grasp the tab 100 of handling portion 10 with the thumb and forefinger upon removing the applicator 1 from the storage container 40. Cavity 400 further comprises a second cavity portion 402 (distal end) for accommodating dry ocular health product OHP, as becomes more evident from the following description.

Storage container 40 is made from a liquid-tight foil, for example an aluminum foil, which does not allow liquid, moisture or vapor to penetrate therethrough, so that storage container 40 comprising cavity 400 can be manufactured through deep-drawing and does not allow liquid, moisture or vapor to enter the primary package which is formed by sealing the cover foil 30 to the storage container 40.

To allow for sealing cover foil 30 to storage container 40, storage container 40 comprises a sealing surface 403 to which surface 300 of cover foil 30 is to be sealed along a sealing portion 404 completely extending around cavity 400. Sealing portion 404 (bounded by the dashed line in FIG. 4 by way of example) is that portion between the dashed line and the edges bounding surface 403 of storage container 40. The rectangular shape of the dashed line may not be the true borderline of the sealing portion 404 but rather is illustrated in FIG. 7 by way of example for the sake of understanding the invention.

As is shown in FIG. 8, storage container 40 with applicator 1 contained therein (as is indicated by the dashed arrow, since applicator 1 is not visible in FIG. 8) is turned upside-down and is then arranged above cover foil 30 on the surface 300 on which dry ocular health product OHP is arranged in a position such that second cavity portion 402 is aligned with dry ocular health product OHP arranged on the surface 300 of cover foil 30. Thereafter, storage container 40 is lowered (see unlabeled arrow in FIG. 8) until sealing surface 403 of storage container 40 contacts surface 300 of cover foil 30, thereby accommodating dry ocular health product OHP in the second portion 402 of the cavity 400. Next, sealing of cover foil 30 along the sealing portion 404 is performed to form the (sealed) primary package.

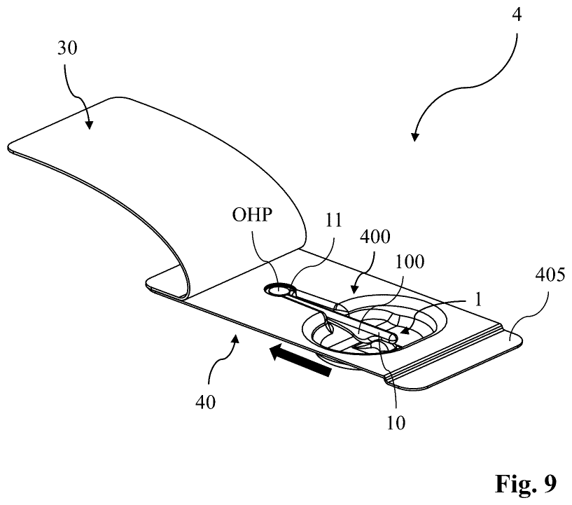

FIG. 9 shows an embodiment of the primary package 4 according to the invention, with the cover foil 30 being partially peeled off of the storage container 40 so as to allow access to the cavity 400 of storage container 40 that contains both the applicator 1 as well as the dry ocular health product OHP (e.g. the polymer gel film). Dry ocular health product OHP adheres to the tip portion 11 (distal end) of applicator 1 due to electrostatic forces, as has been explained in detail above.

Storage container 40 comprises a stepped tab 405, and in the area of the stepped tab 405 cover foil 30 is not sealed to storage container 40. To open the sealed primary package 4 stepped tab 405 may be grasped by the patient with the thumb and the forefinger of one hand, while the non-sealed portion of the cover foil 30 that extends over the stepped tab 405 of the sealed primary package 4 is grasped with the thumb and the forefinger of the other hand. Cover foil 30 may then be peeled off of the storage container 40 in the direction of the unlabeled arrow shown in FIG. 9. However, cover foil 30 is not fully peeled off of the storage container 40. Rather, a portion of cover foil 30 remains sealed to the storage container 40 to avoid inadvertent ejection of dry ocular health product OHP from the cavity 400 due to a jerky movement that may occur when the cover foil 30 is suddenly ripped off of the storage container 40 during complete removal of the cover foil 30.

When the primary package 4 is in the open state shown in FIG. 9, the patient may grasp the tab 100 at the handling portion 10 of applicator 1 and may remove the applicator 1 with the dry ocular health product OHP adhering to the tip portion 11 due to electrostatic forces, as has been explained in detail above. Applicator 1 may then be moved by the patient towards the eye, with the free surface of ocular health product OHP (i.e. that surface of ocular health product OHP facing away from the flat surface 110 of tip portion 11 of applicator 1, see FIG. 2) facing towards the surface of the eyeball of the patient. At the time dry ocular health product OHP is arranged a small distance from the eyeball (still without touching the eye), due to attraction forces caused by the tear film in the eye dry ocular health product OHP releases from tip portion 11 of applicator 1 in a flipping movement towards the eye, and subsequently adheres to the eye of the patient where the dry ocular health product OHP (here: the polymer gel film) dissolves.

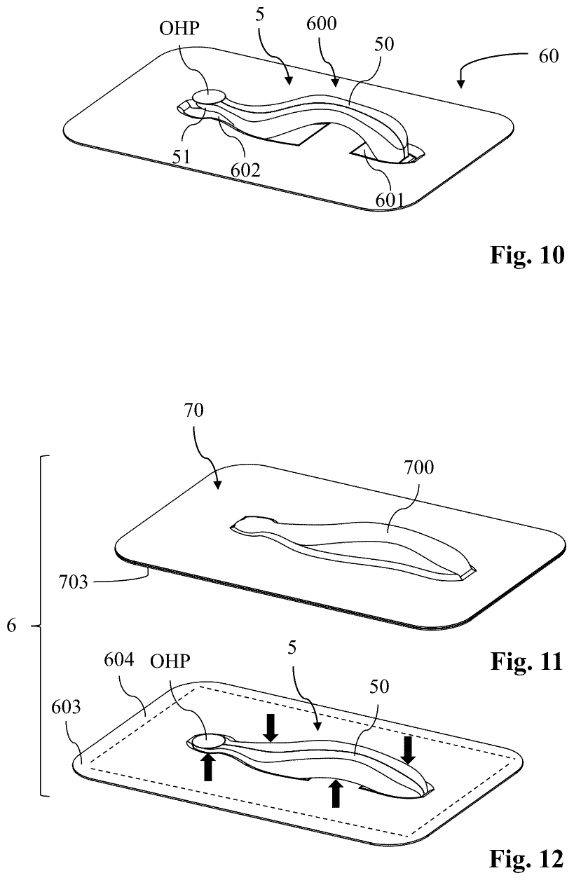

FIG. 10 shows a third embodiment of an applicator 5 according to the invention, the applicator 5 being shown in a relaxed state arranged in a storage container 60 of another embodiment of the primary package according to the invention. Like the first and second embodiments, the third embodiment of the applicator 5 comprises a handling portion 50 (proximal end) for handling the applicator 5. Applicator 5 further comprises a tip portion 51 (distal end) connected to the handling portion 50, for gripping the dry ocular health product OHP and keeping it adhered thereto. In the relaxed state shown in FIG. 10, the applicator 5 with the dry ocular health product OHP adhered to the tip portion 51 thereof may be conveniently taken out of the storage container 60, and the dry ocular health product OHP may be placed on the eyeball of the patient as this has been described further above.

The storage container 60 of primary package 6 contains the applicator 5 and the dry ocular health product OHP in a different state than that shown in FIG. 10, in particular in a state in which they occupy a smaller overall volume. This is more evident from FIG. 11 and FIG. 12. Handling portion 50 of applicator 5 is elastically deformed to a deformed (i.e. tensioned/unrelaxed) state, in which handling portion 50 (proximal end) of applicator 5 is frictionally engaged by inner walls of a first cavity portion 601 of cavity 600 of storage container 60, and in which tip portion 51 of applicator 5 is frictionally engaged by inner walls of a second cavity portion 602 of cavity 600 of storage container 60 as well. FIG. 12 shows the (deformed) state in which the applicator 5 with dry ocular health product OHP adhered to the tip portion 51 thereof is contained in the storage container 60 after removal of the cover foil 70 (see FIG. 11). Once the patient releases the frictional engagement between the applicator and the inner walls of the cavity, the applicator 5 elastically returns to its relaxed state (see FIG. 10), this return to the relaxed state being indicated by the unlabeled arrows shown in FIG. 12.

Like the first and second embodiments, the third embodiment of the applicator 5 may be made of silicone, which is a soft material that is elastically deformable and is easily electrostatically chargeable to make the dry ocular health product adhere to the tip portion 51 thereof.

As can be seen in FIG. 11, the cover foil 70 of this embodiment of the primary package 6 has a cavity 700 formed therein for accommodating those portions of the applicator 5 that protrude upwardly beyond the sealing surface 603 of storage container 60 (even in the deformed state), so that surface 703 cover foil 70 may be sealed to sealing surface 603 of storage container 60 along sealing portion 604 to form the (sealed) fluid-tight primary package 6 containing the applicator 5 and the dry ocular health product OHP.

Embodiments of the invention have been described with the aid of the drawings. However, the invention is not limited to these embodiments, but rather a number of changes and modifications are conceivable without departing from the teaching of the instant invention. By way of example only, in case the handling portion is foldable or compressible (see handling portion 20 shown in FIG. 3) the handling portion may unfold/expand at the time the cover foil is peeled off of the storage container 40 to open the primary package 4. This allows an improved access of the patient to the handling portion which may then be easier to grasp. Other changes or modifications are possible as well. The scope of protection is therefore defined by the appended claims.

1. Applicator (1; 2; 5) for gripping and removing a dry ocular health product (OHP) from a storage container (40; 60) and for placing the removed dry ocular health product (OHP) on the eye of a patient, the applicator comprising:
a handling portion (10; 20; 50) for handling the applicator; and
a tip portion (11; 21; 51) connected to the handling portion (10; 20; 51), for gripping the ocular health product (OHP) from the storage container (40; 60) and for subsequently releasing the gripped ocular health product (OHP) from the tip portion (11; 21; 51) on the eye of the patient, wherein at least the tip portion (11; 21; 51) of the applicator is made from an electrostatically chargeable material to make the dry ocular health product (OHP) adhere to the tip portion (11; 21; 51) due to electrostatic forces exerted by the tip portion (11; 21; 51) of the applicator on the dry ocular health product (OHP), and for subsequently releasing the dry ocular health product (OHP) from the tip portion (11; 21; 51) on the eye of the patient by means of attraction forces caused by a tear film in the eye of the patient.

2. Applicator according to claim 1, wherein the material the applicator (1; 2; 5) is made of is a soft material that is capable of deforming upon contacting the eye.

3. Applicator according to claim 1, wherein the electrostatically chargeable material the tip (11; 21; 51) is made of is silicone.

4. Applicator according to claim 3, wherein the whole applicator (1; 2; 5) is made of silicone.

5. Applicator according to claim 1, wherein the dry ocular health product (OHP) has a flat surface (OHPS) having an ocular health product surface area, and wherein the tip portion (11) of the applicator (1) has a flat surface (110) having tip portion surface area that is substantially congruent with the ocular health product surface area

6. Applicator according to claim 1, wherein the handling portion (20; 50) is elastically deformable in a direction transverse to a direction of the connection of the tip portion (21; 51) and the handling portion (20; 50).

7. A primary package (4; 6) containing a dry ocular health product (OHP) and an applicator (1; 2; 5) according to claim 1, the primary package (4; 6) comprising a fluid-tight storage container (40; 60) having a cavity (400; 600) accommodating the dry ocular health product (OHP) and the applicator (1; 2; 5), the primary package (4; 6) further comprising a fluid-tight cover foil (30; 70) sealed along a sealing portion (404; 604) of the storage container (40; 60), the sealing portion (404; 604) completely extending around the cavity (400; 600) accommodating the dry ocular health product (OHP) and the applicator (1; 2; 5), wherein the dry ocular health product (OHP) is arranged in the cavity (400; 600) at the location where the tip portion (11; 21; 51) of the applicator (1; 2; 5) is arranged.

8. A primary package according to claim 7, wherein the dry ocular health product (OHP) is a dried polymer gel film to be dissolved upon being placed on the eye.

9. A primary package according to claim 7, wherein the fluid-tight storage container (40; 60) is made of a fluid-tight foil, for example an aluminum foil, and the cavity (400; 600) is formed in the fluid-tight foil through deep-drawing or thermoforming, or through combinations thereof.

10. A primary package according to claim 7, wherein the dry ocular health product (OHP) is transparent or translucent, and wherein the fluid-tight cover foil (30; 70) is opaque and colored.

11. A method of manufacturing a primary package (4) according to claim 7, the method comprising:
providing an applicator (1; 2; 5) for gripping and removing a dry ocular health product (OHP) from a fluid-tight storage container (40; 60), and for placing the removed dry ocular health product (OHP) on the eye of a patient, the applicator (1; 2; 5) comprising a handling portion (10; 20; 50) for handling the applicator and a tip portion (11; 21; 51) connected to the handling portion (10; 20; 50), the tip portion (11; 21; 51) for gripping the dry ocular health product (OHP);
providing a fluid-tight storage container (40; 60) comprising a cavity (400; 600) for accommodating the applicator (1; 2; 5) and the dry ocular health product (OHP), and having a sealing portion (404; 604) completely extending around the cavity (400; 600);
providing a fluid-tight cover foil (30; 70) to be sealed to the storage container (40; 60) along the sealing portion (404; 604), with a surface (300; 703) of the cover foil (30; 70) to be sealed to the storage container (40; 60) along the sealing portion (404; 604) facing upwards;
dispensing a predetermined amount of a liquid ocular health product (LOHP) on a predetermined area of the surface (300; 703) of the cover foil (30; 70) that is to be sealed to the storage container (40; 60) along the sealing portion (404; 604);
drying the predetermined amount of the liquid ocular health product (LOHP) to obtain a dry ocular health product (OHP) that is arranged on the predetermined area of the surface (300; 703) of the cover foil (30; 70) that is to be sealed to the storage container (40; 60) along the sealing portion (404);
placing the applicator (1; 2; 5) in the cavity (400; 600) of the storage container (40; 60) in a manner such that the applicator (1; 2; 5) is contained therein frictionally engaged by inner walls of the cavity (400; 600);
placing the storage container (40; 60) with the frictionally engaged applicator (1; 2; 5) contained therein on the cover foil (30; 70), with the sealing surface (403; 603) of the storage container (40; 60) facing towards that surface (300; 703) of the cover foil (30; 70) on which the dry ocular health product (OHP) is arranged, and with the cavity (400; 600) arranged to accommodate the dry ocular health product (OHP) at the location where the tip portion (11; 21; 51) of the applicator (1; 2; 5) is arranged; and
sealing the cover foil (30; 70) to the storage container (40; 60) along the sealing portion (404; 604) to form the primary package (4; 6).

12. Method according to claim 11, wherein the applicator is an applicator (2) having a handling portion (20; 50) that is elastically deformable in a direction transverse to a direction of the connection of the tip portion (21; 51) and the handling portion (20; 50), and wherein the method further comprises the step of folding or compressing the handling portion (20; 50) of the applicator (2; 5) during placing the storage container (40; 60) with the frictionally engaged applicator (2; 5) contained therein on the cover foil (30; 70).
